(19) United States
(12) Patent Application Publication
(10) Pub. No.: US 2002/0177625 A1
(43) Pub. Date: Nov. 28, 2002

(54) Class of glaucoma drugs to enhance aqueous humor outflow and lower intra-ocular pressure

(75) Inventors: Martha E. O'Donnell, Davis, CA (US); Fitz-Roy E. Curry, Davis, CA (US); James D. Brandt, Sacramento, CA (US)

Correspondence Address: MI K. KIM, Fish & Richardson P.C., Suite 500, 4350 La Jolla Village Drive, San Diego, CA, 92122, US

(73) Assignee: The Regents of the University of California, a California Corporation

(21) Appl. No.: 10/134,099

(22) Filed: Apr. 26, 2002

Related U.S. Application Data
(60) Provisional application No. 60/287,194, filed on Apr. 28, 2001.

(51) Int. Cl. A61K 31/192

(52) U.S. Cl. 514/569

(57) ABSTRACT

Elevated intra-ocular pressure is reduced by administration directly to the eye of compounds that activate the Cl and K channels of the trabecular meshwork and/or Schlemm's canal endothelial cells of the mammalian eyes. Intra-ocular pressure may further be reduced by the co-administration of compounds that inhibit function of a Na⁺—K⁺—2Cl⁻ co-transporter mechanism of the trabecular meshwork and/or Schlemm's canal endothelial cells. These compounds are useful in treatment of diseases of the eye associated with elevated intra-ocular pressure, such as ocular hypertension and glaucoma. A screening method is provided to discover additional compounds with utility for lowering intra-ocular pressure by substantially activating the Cl and K channels.

### FIELD OF THE INVENTION

This invention relates to methods for reducing the intra-ocular pressure of the eye by enhancing aqueous humor outflow and to a method for screening compounds that reduce intra-ocular pressure.

### BACKGROUND OF THE INVENTION

In glaucoma, a leading cause of blindness, the optic nerve is damaged through a poorly-understood interaction of elevated intra-ocular pressure (IOP) and patient predisposition to the disease. Mechanisms that regulate aqueous humor outflow and intra-ocular pressure are thought to be defective in such a manner that an increase in IOP result.

The anterior chamber of the eye is bathed with aqueous humor, formed continuously by the ciliary body. Production of aqueous humor occurs along the surface of these ciliary processes (pars plicata), themselves covered by a double layer of epithelial cells consisting of a pigmented and non-pigmented layer situated with their apical surfaces juxtaposed. They function in tandem to produce transepithelial secretion of NaCl and water in movement from the blood to the aqueous humor. The rate of aqueous humor production is quite high relative to other types of epithelia that function in vectorial transport of water and electrolytes. The aqueous humor production and drainage mechanisms work to replace the entire volume of aqueous humor every 100 minutes. Thus, an effective drainage pathway to accommodate this rate of fluid production is essential for maintenance of normal intra-ocular pressure.

Aqueous humor moves by bulk flow from its site of production in the posterior chamber through the pupillary aperture and into the anterior chamber. It subsequently exits the anterior chamber via one of two routes. The majority of outflow in the healthy human eye occurs at the anterior chamber angle, where aqueous humor passes through the trabecular meshwork and into the Canal of Schlemm (also known as Schlemm's canal), from where it joins the general venous drainage of the eye. A second outflow pathway is via the uveoscleral route, although this appears to be a minor (≅20%) pathway in the normal human eye. A homeostatic balance of aqueous humor production and drainage allows intra-ocular pressure to be maintained within narrow limits in the normal eye.

Focusing on the primary pathway, after passing through the trabecular meshwork, aqueous matter crosses the endothelial cells of the Canal of Schlemm. In this manner, trabecular meshwork cells and Canal of Schlemm endothelial cells are thought to comprise the cells of the primary outflow pathway of the eye. The trabecular meshwork is suspended between the corneal endothelium and the ciliary body face and is comprised of a series of parallel layers of thin, flat, branching and interlocking bands termed trabeculae. The inner portion of the trabecular meshwork (closest to the iris root and ciliary body) is called the uveal meshwork, whereas the outer portion (closest to the Canal of Schlemm) is called the corneoscleral or juxtacanalicular meshwork. The uveal meshwork trabeculae measure approximately 4 μm in diameter, consist of a single layer of cells surrounding a collagen core, and are arranged in layers which are interconnected. The spaces between these trabeculae are irregular and range from about 25 μm to about 75 μm in size. The trabeculae of the corneoscleral meshwork resemble broad, flat endothelial sheets about 3 μm thick and up to about 20 μm long. The spaces between these trabeculae are smaller than in the uveal meshwork and more convoluted. As the lamellae approach the Canal of Schlemm, the spaces between the trabeculae decrease to about 2 μm. The resistance to aqueous humor outflow through the trabecular meshwork has been reported to reside primarily in the juxtacanalicular meshwork. At this site two cell types are found: trabecular meshwork cells and also endothelial cells of the inner wall of Schlemm's canal.

In contrast to the current level of knowledge regarding cellular processes responsible for aqueous humor production by the ciliary body, relatively little is known about the cellular mechanisms in the trabecular meshwork that determine the rate of aqueous outflow. Pinocytotic vesicles are observed in the juxtacanalicular meshwork and the inner wall of Schlemm's Canal. The function of these vesicles remains unknown, but some investigators have suggested that the bulk flow of aqueous humor through the meshwork cannot be accounted for by flow through the intercellular spaces and that these vesicles play a central role in outflow regulation. Evidence has been provided that cytoskeleton-mediated changes in trabecular meshwork cell shape modulate aqueous outflow. The extracellular matrix surrounding the trabeculae is thought to contribute to outflow resistance, perhaps by interactions with proteins contained in the aqueous humor. Abnormalities in this extracellular matrix may contribute to the increased outflow resistance seen in corticosteroid-induced glaucoma. Investigators evaluating both normal physiology and drug effects have provided evidence that changes in cell shape (as distinct from cell volume) may be involved in outflow regulation. Trabecular meshwork cells have been shown to possess actin and myosin filaments and to contract in response to some agents. It has been speculated that changes in trabecular meshwork cell volume (as distinct from cell shape) may participate in the regulation of aqueous outflow facility. The studies, as discussed herein, support such views.

It is well recognized that regulation of aqueous humor outflow through the trabecular meshwork is critically important for maintenance of an appropriate intra-ocular pressure; and that in disease states such as ocular hypertension and glaucoma, this regulation appears to be defective. For instance, U.S. Pat. No. 4,757,089 teaches a method for increasing aqueous humor outflow by topical or intracameral administration of ethacrynic acid, or an analog thereof, to treat glaucoma. It is also known that ethacrynic acid increases water flux across the walls of perfused microvessels and inhibits Na⁺—K⁺—2Cl⁻ co-transport activity of avian erythrocytes, although the mechanisms by which these phenomena occur have not been elucidated. For instance, phenoxyacetic acids inhibit NaCl reabsorption in the thick ascending limb of the loop of Henle screening test; but its effect was exerted from both epithelial sides, rather than from the luminal side as with the class of loop diuretics, and it led to a depolarization of the membrane voltage. This effect is compatible with an inhibitory action at the level of mitochondrial ATP production rather than an inhibition of the Na⁺—K⁺—2Cl⁻ co-transporter.

In addition to regulation of aqueous outflow, trabecular meshwork cells are thought to serve an immunologic function as they phagocytize antigens in the anterior chamber of the eye as they pass through the trabecular meshwork. It has been hypothesized that the cells then migrate out of the meshwork into the Canal of Schlemm to enter the systemic circulation and act as antigen presenting cells to trigger the production of antibodies to the phagocytized antigen. In at least one form of glaucoma (pigmentary), this phagocytotic function is thought to be overwhelmed, resulting in increased resistance to aqueous outflow. The endothelial cells lining the Canal of Schlemm also appear to contribute to the resistance to outflow in the normal eye.

A number of hormones and neurotransmitters have been documented to decrease intra-ocular pressure by modulating aqueous production or outflow. Studies employing a human eye perfusion model have shown that epinephrine, via an apparent β-adrenergic effect upon the uveo-scleral pathway, increases the facility of outflow. Nitrovasodilators have been found to increase outflow facility and decrease intra-ocular pressure in monkey eye. Similarly, atrial natriuretic peptide decreases intra-ocular pressure in monkey eyes and increases aqueous humor production. In addition to these hormones and neurotransmitters, ethacrynic acid has been shown to increase aqueous outflow and decrease intra-ocular pressure by modulating aqueous inflow and outflow. Elevations of norepinephrine concentration in the aqueous humor resulting from cervical sympathetic nerve stimulation cause an increase in intra-ocular pressure of rabbit eye in situ by a mechanism that appears to involve an α-adrenergic effect. Similarly, topical administration of vasopressin to the eye has been shown to increase intra-ocular pressure and decrease facility of outflow in both normal and glaucomatous human eyes. A local renin-angiotensin system resides in the eye, and inhibition of angiotensin converting enzyme causes a decrease of intra-ocular pressure. In contrast to these rapidly-acting agents, administration of the glucocorticoid dexamethasone increases resistance to outflow over a slower time course of hours and days, an effect that has been postulated to occur in the expression of extracellular matrix.

Despite the large amount of work that has been done in the area of aqueous outflow regulation, more information leading to a better understanding of the regulation and to assist in the discovery of better methods of regulating intra-ocular pressure to treat diseases such as glaucoma is needed.

In general, the glaucomas comprise a heterogeneous group of eye diseases in which elevated IOP causes damage and atrophy of the optic nerve, resulting in vision loss. The underlying cause of the elevated IOP can be grossly divided into two pathophysiologic scenarios in which the drainage pathways are either physically closed off (as in the various forms of angle-closure glaucoma) or in which the drainage pathways appear anatomically normal but are physiologically dysfunctional (as in the various forms of open-angle glaucoma). Angle-closure glaucoma is nearly always a medical and/or surgical emergency, in which pharmacologic intervention is essential in controlling an acute attack, but in which the long-range management is usually surgical in nature. Primary Open Angle Glaucoma (POAG), on the other hand, has a gradual, symptomless onset and is usually treated with chronic drug therapy. POAG is the most common form of glaucoma, comprising 80% of newly-diagnosed cases in the United States and is the leading cause of blindness among African Americans.

Drugs currently used to treat glaucoma can be divided into those that reduce aqueous humor inflow and those that enhance aqueous humor outflow. The most commonly-prescribed drugs at present are the β-adrenergic antagonists, which reduce aqueous humor inflow through an unknown effect on the ciliary body. Other drugs that reduce aqueous inflow include inhibitors of carbonic anhydrase (e.g., acetazolamide and methazolamide) and the alpha-adrenergic agonist apraclonidine. Both of these drug classes exert their clinical effects through a poorly-understood action on the ciliary body. Each of these drugs, although effective in many patients, is poorly tolerated in some because of profound and occasionally life-threatening systemic adverse effects.

Drugs that enhance aqueous humor outflow from the eye include miotics and the adrenergic agonists. The miotics exert a mechanical effect on the longitudinal muscle of the ciliary body and thus pull open the trabecular meshwork. They comprise both direct-acting parasympathomimetic agents (e.g., pilocarpine and carbachol) and indirect-acting parasympathomimetic agents (e.g., echothiopate). Miotic agents are highly effective in lowering IOP but have significant adverse effects, including chronic miosis, decreased visual acuity, painful accommodative spasm and risk of retinal detachment. Adrenergic agonists (e.g., epinephrine and dipivefrin) act on the uveoscleral outflow tract to enhance outflow through a mechanism that remains poorly understood. These drugs have perhaps the best safety profile of the compounds presently used to treat glaucoma, but are among the least effective in their IOP-lowering effect.

Accordingly, the need exists for new and better methods of lowering intra-ocular pressure, particularly in the treatment of one of the leading causes of blindness, glaucoma.

### SUMMARY OF THE INVENTION

The inventors have conducted studies that provide support for a mechanism of regulating the aqueous outflow in the trabecular meshwork cells and Schlemm's canal endothelial cells. These studies demonstrate that in this mechanism, the Na—K—Cl co-transporter works in conjunction with K and Cl channels to regulate intracellular volume of these cells, thereby, regulating aqueous outflow facility and affecting intra-ocular pressure.

The trabecular meshwork (TM) and Canal of Schlemm (SC) endothelial cells are thought to be functionally similar to the vascular endothelium, in that they both present barriers to solute and water flux. Because a relatively large volume of aqueous humor traverses the TM and SC cells each day, the cells of the trabecular meshwork and Canal of Schlemm must be equipped with mechanisms that allow them to appropriately respond to the ever-changing local environment of the anterior chamber. The studies further indicate that intracellular volume of outflow pathway cells is an important determinant of outflow facility.

In previous studies, it has been shown that the Na-K-Cl co-transporter of TM cells functions to regulate intracellular volume, described in U.S. patent application Ser. No. 08/568,389, filed Dec. 6, 1995, issued as U.S. Pat. No. 5,763,491; U.S. patent application Ser. No. 08/353,442, filed Dec. 9, 1994, issued as U.S. Pat. Nos. 5,585,401; and 09/093,961, filed Jun. 8, 1998; the contents of which are herein incorporated by reference in their entirety. It has now been discovered that Na—K—Cl co-transporters are also found in SC cells and that they possess Na—K—Cl activity similar to that of co-transporters in TM cells.

Furthermore, in both TM and SC cells, the co-transporter mediates volume recovery following hypertonicity-induced cell shrinkage (the regulatory volume increase, RVI) and also contributes to maintenance of steady state volume by mediating net influx of Na, K and Cl into the cells (which tends to increase cell volume). Blocking co-transporter activity causes the TM or SC cells to shrink, indicating that the co-transporter normally offsets ion efflux through other pathways, thereby maintaining the desired cell volume. Studies conducted herein support the hypothesis that Cl channels and K channels comprise the ion efflux pathway that normally balances co-transporter activity under steady state conditions. They also support the hypothesis that Cl and K channels mediate the regulatory volume decrease (RVD) which decreases TM cell volume to normal resting levels following hypotonicity-induced cell swelling. Thus, as shown in FIG. 1, it may be the combined actions of Na—K—Cl co-transporter and Cl and K channels that determine the steady state intracellular volume of TM and SC cells. Alteration of steady state volume by changes in co-transport activity and/or Cl and K channel activity is further predicted to alter outflow facility.

These volume-regulatory ion flux pathways are not important simply for restoring volume when the cells encounter changes in extracellular tonicity. The data suggest that hormones and neurotransmitters, which appear to be found in the anterior chamber, can alter Na—K—Cl co-transporter activity to change intracellular volume which, in turn, appears to be a determinant of outflow facility (Al-Aswad, L. A. et al., Invest. Ophthalmol. Vis. Sci., 40: 1695-1701, 1999; O'Donnell, M. E. et al., Amer. J. of Physiology 268: C1067-C1074, 1995; Putney, L. K. et al., Invest. Ophthalmol. Vis. Sci., 40: 425-434, 1999. These agents may also alter activity of volume-regulating Cl and K channels. It has been shown in other cell types that these volume-regulating mechanisms are also important for maintaining resting volume when metabolic changes in the cell occur (which can alter the number of intracellular osmolytes and cause the cells to shrink or swell). It should be noted that at least one type of volume-sensitive Cl channel (the VSOAC channel) can also be activated by membrane stretch. This would suggest that if VSOAC Cl channels are present in TM and SC cells, they could also be activated to decrease volume when the cells are stretched, as may occur when intra-ocular pressure increases. Thus, such a stretch-activated decrease in TM and/or SC cell volume would serve to increase aqueous outflow facility.

In one aspect, the present invention provides a method of increasing aqueous humor outflow in the eye of a mammalian patient, such as a human, by administering an effective amount of a composition that includes a compound that inhibits a Na⁺—K⁺—2Cl⁻ co-transporter in the SC cells. In another aspect, the present invention provides a method for increasing aqueous humor outflow in the eye by administering an effective amount of a composition that includes a compound that activates a Cl channel and/or a K channel in the TM and/or SC cells. In yet another aspect, the present invention provides a method of increasing aqueous humor outflow in the eye by administering a compound that inhibits a Na⁺—K⁺—2Cl⁻ co-transporter in the TM and/or SC cells and a compound that activates a Cl channel and/or a K channel in the TM and/or SC cells. The compound that inhibits the co-transporter may or may not be the same compound that activates the Cl and/or K channels.

In one embodiment of the invention, the method for reducing intra-ocular pressure uses a new class of compounds, hitherto known as nonsteroidal anti-inflammatory agents, due to their ability to activate the Cl and/or K channels of the TM and SC cells. Examples of nonsteroidal anti-inflammatory agents include niflumic acid and flufenamic acid. In another embodiment, the use of compounds that activate the Cl and/or K channels may also include the use of compounds that substantially inhibit operation of the Na⁺—K⁺—2C⁻ co-transporter mechanism, an example of which includes the use of high ceiling diuretics, also known as loop diuretics, such as benzmetamide, bumetamide, furosemide, torasemide, and piretamide. The administration of Cl or K channel activating compound may be before, after, or during the administration of the Na⁺—K⁺—2Cl⁻ co-transporter inhibiting compound.

The composition having an effective amount of an outflow-increasing compound may be applied either topically, by corneal iontophoresis, or by intracameral microinjection into the anterior chamber of the eye. The delivery of these compounds may be enhanced by the use of an erodible or sustained release ocular insert device. In one embodiment, the composition to be administered by microinjection may include 0.025% benzalkonium chloride. In another embodiment, the composition to be topically applied may include a lipophilic or amphipathic derivative of the outflow-increasing compound, examples of the compound include, but are not limited to, niflumic acid, flufenamic acid, benzmetamide, bumetamide, furosemide, torasemide, and piretamide. The topically applied compositions may also include compounds that enhance corneal penetration. For example, in one preferred embodiment, the compound may have a octanol:water coefficient of at least 0.005. In another preferred embodiment, the compound may have an octanol:water coefficient of at least 0.01.

The methods of the invention contemplated herein are used to increase the outflow of ocular fluids, thereby lowering intra-ocular pressure. The compounds disclosed herein are useful in the treatment of diseases of the eye associated with elevated intra-ocular pressure, such as ocular hypertension and glaucoma.

A screening method is also provided to discover additional compounds with utility for lowering intra-ocular pressure by substantially activating the K and/or Cl channels of the TM or SC cells. For example, a method for screening compounds for utility in increasing aqueous humor outflow can include the steps of contacting Schlemm's canal endothelial cells or trabecular meshwork cells with a compound in the presence and absence of K and/or Cl channel blockers. Then the cells are observed for physiological changes in the presence of K and/or Cl channel blockers and in the absence of K and/or Cl channel blockers, whereby the changes are indicative of the compound's use in regulating aqueous humor outflow. The observed physiological change can be a change in the conductance of the cells or a change in the volume of the cells. The screening method can also include a further step of observing the compound's effect on Na—K—Cl co-transporter activity.

### BRIEF DESCRIPTION OF THE DRAWINGS

FIG. 1 is a schematic diagram of a proposed role of Na—K—Cl co-transport and K and Cl channels in regulation of TM cell and SC cell volume.

FIG. 2A is a bar graph of the Na—K—Cl co-transport activity in human TM cells.

FIG. 2B is a diagram of a western blot shown the presence of Na—K—Cl protein in freshly isolated human TM and cultured TM cells.

FIGS. 3A-B show line graphs of relative cell volumes in response to differing conditions.

FIG. 4 is a bar graph of the Na—K—Cl co-transport activity in human SC cells.

FIGS. 5A-B show the cell volume and Na—K—Cl co-transport activity, respectively, of normal and glaucomatous human TM cells.

FIGS. 6A-B show the conductance of volume-sensitive Cl channels in human TM cells.

FIGS. 7A-D shows relative cell volumes of human TM cells by Cl and K channel blockers.

FIG. 8 shows swelling-activated ion conductance in human TM cells.

FIGS. 9A-B show the effects of niflumic acid on Na—K—Cl co-transport activity and relative cell volume, respectively, of human TM cells.

FIGS. 10A-B show the effect of niflumic acid on the ion conductance in human TM cells.

### DETAILED DESCRIPTION OF THE INVENTION

It has been discovered that mammalian trabecular meshwork (TM) cells, e.g., bovine and human, possess a Na⁺—K⁺—2Cl⁻ co-transport system that works in conjunction with Cl and K channels to regulate the intra-ocular pressure (IOP). With the discovery of Na⁺—K⁺—2Cl⁻ co-transport system in Schlemm's canal (SC) endothelial cells, it is believed that SC cells also possess Cl and K channels that work in conjunction with the Na⁺—K⁺—2Cl⁻ co-transport system to regulate the IOP.

The present invention provides a method for increasing aqueous humor outflow in the eye of a human or other mammal by administration to the eye of an effective amount of a compound that substantially activates K and/or Cl channels in the trabecular meshwork (TM) cells and/or Schlemm's canal (SC) endothelial cells. The present invention also provides a method for increasing aqueous humor outflow in the eye of a human or other mammal by administration to the eye of an effective amount of a compound that substantially activates K and/or Cl channels and an effective amount of a compound that substantially inhibits the Na⁺—K⁺—2Cl⁻ co-transporter mechanism in TM and/or SC cells.

As used herein the terms “K channel” and “Cl channel” refer generically to the structures and mechanisms that actively or passively transport ions such as K and Cl into and out of cells.

“Na—K—Cl transporter” refers generically to the co-transporter systems, but does not specify the particular stoichiometry of transport in the system described. On the other hand, as used herein the term Na⁺—K⁺—2Cl⁻ co-transport system refers only to those co-transport systems having the indicated stoichiometry.

Cell volume regulating processes vary with the cell type; however in virtually all cells, acute cell volume regulation is carried out by two types of ion flux pathways acting in concert: ion influx pathways that work to increase cell volume and ion efflux pathways that work to decrease cell volume.

The two most common types of volume-increasing ion flux mechanisms are: 1) Na/H exchange plus Cl/HCO₃ exchange working together to mediate net uptake of NaCl (with water following the ions into the cell); and 2) Na—K—Cl co-transport which mediates net uptake of Na, K and Cl (with water following). Previous studies have demonstrated that the Na—K—Cl co-transporter plays this role in trabecular meshwork cells as well as in vascular endothelial cells (O'Donnell, M. E. et al., Amer. J. of Physiology 264: C1316-C1326, 1993). It has now been discovered that the Na—K—Cl co-transporter plays this role in Schlemm's canal endothelial cells. Evidence has also been provided that Na—K—Cl co-transport and the Na/K pump act in concert to bring about the vectorial transport (Dong, et al., Invest. Ophthalmol. Vis. Sci., 35:1660, 1994).

The Na-K-Cl co-transport system is an electroneutral symport mechanism that moves 1Na, 1K, and 2Cl ions across the plasma membrane of cells. Distinguishing features of the co-transporter are that it is: 1) inhibited by “loop” diuretics such as bumetamide and furosemide; 2) highly selective for Na, K, and Cl (with the exception that it carries Rb as well as K); and 3) requires the presence of all three transported ion species in order to operate. Thus, activity of the co-transporter can be assessed as a bumetamide-sensitive, Na- and Cl-dependent K influx, using ⁸⁶Rb as a tracer for K.

Whereas these kinetic and pharmacological features of Na—K—Cl co-transport are quite constant among different cell types, the regulation of co-transport is heterogeneous. In vascular endothelial cells, trabecular meshwork cells, and Schlemm's canal endothelial cells, the co-transporter is stimulated by elevation of intracellular Ca (and by hormones that increase Ca such as vasopressin and angiotensin II) but inhibited by elevation of either cyclic AMP or cyclic GMP (or by norepinephrine or atrial natriuretic peptide). The co-transporter is also stimulated by hypertonic media (i.e., cell shrinkage) and inhibited by hypotonic media (i.e., cell swelling). In cells that utilize Na—K—Cl co-transport to volume regulate, cell shrinkage stimulates the co-transporter, which in turn mediates a net uptake of Na, K, and Cl into the cell. As water re-enters the cell with the transported ions, the cell re-swells. This compensatory increase in cell volume is called a regulatory volume increase (RVI). Exposure of cells to hypotonic media causes cells to swell rapidly as water enters the intracellular space, followed by a compensatory decrease in cell volume, the regulatory volume decrease (RVD), which appears to be mediated by net efflux of ions through transporters separate from the Na—K—Cl co-transporter (e.g., K—Cl co-transport and K and Cl channels). Simply inhibiting the co-transporter by bumetamide can make some cells shrink; this includes vascular endothelial cells, trabecular meshwork cells, and Schlemm's canal endothelial cells. Further, hormone modulation of co-transporter activity can drive changes in cell volume even under isosmotic conditions.

Cell volume cannot be regulated and/or maintained by volume-increasing ion influx pathways alone, i.e., volume-decreasing ion efflux pathways also contribute to this process. In aortic endothelial cells and trabecular meshwork cells, for example, inhibition of the co-transporter causes the cells to shrink, indicating that the co-transporter normally works to offset ion efflux pathways. When cells are swollen, volume-sensitive ion efflux pathways are activated to effect a regulatory volume decrease (RVD). Pathways responsible for this phenomenon can include K—Cl co-transport and K and Cl channels, depending on the cell type. Data is provided to support a role for Cl and K channels in volume regulation of trabecular meshwork and Schlemm's canal endothelial cells.

Volume-regulatory Cl channels are known to vary with cell type. Although a number of Cl channels have been described, they generally fall into a few classes. These include: 1) VSOAC, the volume-activated, outward rectifying Cl channel which can also carry organic anions; 2) ClC₂, thought to be a ubiquitously expressed “housekeeping” Cl channel activated by cell swelling and by hyperpolarization; 3) I_{Cln}, the epithelial Na channel which is also volume-sensitive (and which may be the same protein as VSOAC; and 4) the Maxi anion channel, activated by both cell swelling and by membrane stretch. As its name indicates, this channel has the largest single-channel conductance of the Cl channels. Each of these channels exhibits characteristic features related to their ability for anion selectivity (e.g., selectivity to Cl⁻, SCN⁻, I⁻, NO₃⁻, Br⁻, F⁻, gluconate, cyclamate, acetate and HCO₃⁻). They also exhibit different sensitivities to Cl channel blockers (e.g., NPPB, DPC, DDF and the stilbenes SITS and DIDS) and also different sensitivity to inhibition or activation by agents other than swelling (e.g., hyperpolarization, membrane stretch, extracellular nucleotides and cytoskeletal disrupters). These channels also exhibit characteristic current/voltage relationship plots and volume-sensitivities, but the specific characteristics seem to vary greatly among tissues and cell types.

At least two types of volume regulatory K channels have been found to serve this function in various cells: 1) swelling-activated K channels; and 2) Ca-activated K channels (also known as BK channels). For the latter, cell swelling causes an increase in intracellular [Ca], which then activates the Ca-activated K channels. The elevation of intracellular [Ca] can occur via a variety of signaling pathways, depending on the cell type. Intracellular [Ca] can increase via release from intracellular stores or via entry from the extracellular solution via stretch-activated channels.

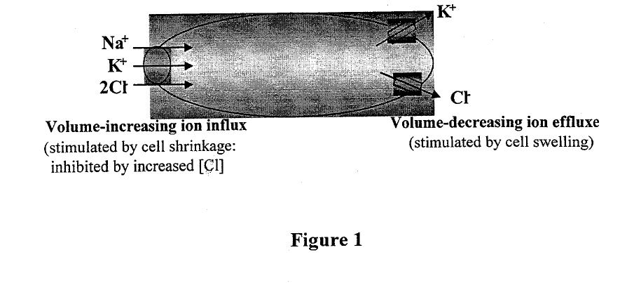

FIG. 1 shows a schematic diagram of the possible influx/efflux system of TM and SC cells. The Na—K—Cl co-transporter, K channel and Cl channel function to regulate intracellular volume. The co-transporter mediates volume recovery following hypertonicity-induced cell shrinkage and it also contributes to maintenance of intracellular volume under steady state conditions. In this regard, blocking co-transporter activity causes the TM and SC cells to shrink, indicating that the co-transporter normally offsets ion efflux through other pathways. The Cl channels and K channels may comprise the ion efflux pathway normally balancing co-transporter-mediated ion influx under steady state conditions. Thus, as shown in FIG. 1, the combined actions of co-transporter and Cl and K channels may determine the steady state intracellular volume of TM and/or SC cells. Accordingly, alteration of TM and/or SC volume by changes in co-transport activity and/or channel activity is predicted to alter outflow facility.

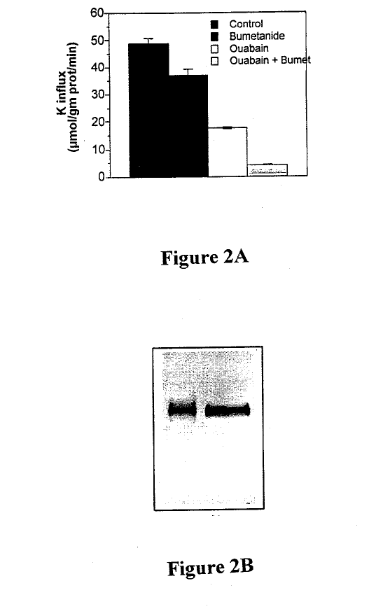

FIG. 2A is a bar graph showing Na—K—Cl co-transporter activity in cultured TM cells. In this figure, it can be seen that the co-transport inhibitor bumetamide blocks a substantial portion of the total K influx observed in the TM cells (compare dark gray and black bars; control vs. bumetamide). This bumetamide-inhibitable, or bumetamide-sensitive, K influx is also observed when the Na/K pump inhibitor ouabain is present (i.e., the difference between white and light gray bars; ouabain vs. ouabain+bumetamide). When either Na or Cl is omitted from the assay medium, the bumetamide-sensitive K influx is abolished. This Na- and Cl-dependent bumetamide-sensitive K influx is, by definition, Na—K—Cl co-transport activity. These findings, thus, demonstrate the presence of Na—K—Cl co-transport activity in human TM cells and bovine TM cells.

If the Na—K—Cl co-transporter is important in trabecular meshwork function then it should be present not only in cultured TM cells but also in freshly isolated TM. This is indeed the case, as shown in FIG. 2B. FIG. 2B is a picture of a western blot showing protein bands of approximately 170 kDa appearing in both cultured human TM cells and freshly isolated human TM cells. Thus, the presence of the co-transporter in cultured TM cells indicates that it is not simply a culturing-induced phenomenon but rather, a protein important for TM cell function in vivo.

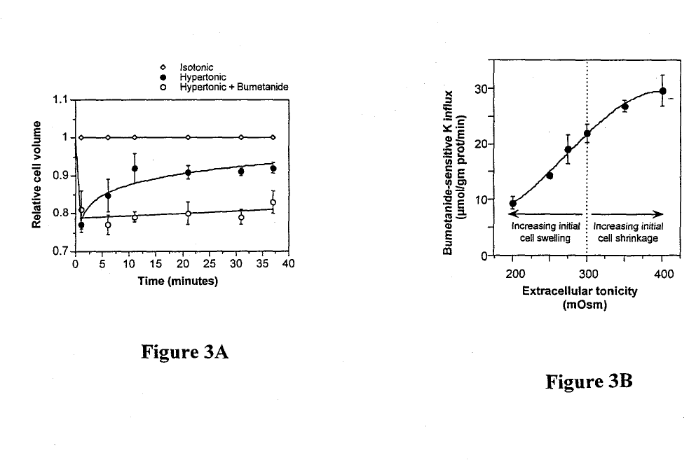

Na—K—Cl co-transporter has been found to participate in the regulation of TM intracellular volume. The co-transporter: 1) helps TM cells restore their volume following hypertonic shrinkage; 2) maintains TM cell volume under isotonic conditions by offsetting ion efflux pathways that tend to shrink the cells; and 3) mediates hormone-driven changes in TM cell volume. A classic test of whether a putative volume-regulatory ion transporter does participate in regulation of cell volume is to perturb cell volume and determine whether the subsequent cell volume recovery is dependent on the activity of that transporter. FIG. 3A is a line graph that shows that when TM cells are exposed to hypertonic medium, they shrink rapidly, as expected (as water is drawn out of the cell). Subsequently, the cells begin to increase their volume again toward control isotonic level (pre-hypertonic shrinkage volume). If the cells are exposed to hypertonic media containing bumetamide to inhibit activity of the Na—K—Cl co-transporter, their volume recovery is abolished. This indicates that co-ransporter activity is vital for the volume recovery, called a regulatory volume increase (RVI).

FIG. 3B is a line graph that shows that TM cell co-transporter is stimulated by hypertonic media and inhibited by hypotonic media. This is as predicted for a transporter that is activated during cell shrinkage to re-swell the cells to normal volume. Inhibition of the co-transporter by hypotonic media is also expected, since typically ion pathways involved in the RVI are shut off when cells swell while ion pathways involved in reducing cell volume (such as K and Cl channels) are activated. The studies show that inhibiting the co-transporter under isotonic conditions causes a significant decrease in cell volume. For example, it has been found that 30 and 60 minute exposures of human TM cells to 10 μM bumetamide caused 12±4% and 25±6% reductions in intracellular volume, respectively. This phenomenon, which occurs in both bovine and human TM cells, indicates that the cells require basal co-transporter activity just to maintain normal resting volume and that, when it is inhibited, volume decreasing ion efflux pathways cause the cells to shrink. Hormones and intracellular regulators which inhibit activity of the co-transporter also reduce TM cell volume, e.g., norepinephrine and cyclic AMP, whereas agents that stimulate activity of the co-transporter increase TM cell volume, e.g. phorbol esters in bovine TM cells. Studies have also shown that the co-transporter mediates hormone-driven changes in cell volume of vascular endothelial cells as well, such that vasopressin stimulates the co-transporter and increases cell volume in a manner blocked by bumetamide, whereas norepinephrine inhibits the co-transporter and reduces cell volume (O'Donnell, M. E., Amer. J. of Physiology, 257: C36-C44, 1989).

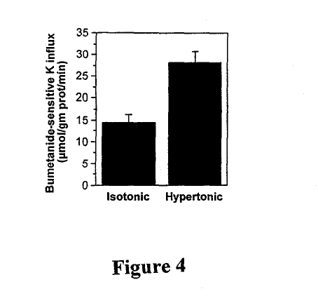

As previously discussed, Canal of Schlemm endothelial cells (SC cells), along with trabecular meshwork cells, comprise the cells of the outflow pathway. However, the regulatory mechanism of cell volume regulation of SC cells had been unknown. Studies in extraocular vascular endothelial cells indicate that intracellular volume of the endothelial cells is a determinant of barrier permeability such that conditions which shrink the endothelial cells increase permeability. The intracellular volume of SC cells, along with the volume of TM cells, may well contribute to determining outflow facility. Studies have also shown that in extraocular vascular endothelial cells, the Na—K—Cl co-transporter plays a pivotal role in regulation of intracellular volume, much as it does in TM cells. In FIG. 4, the studies show robust co-transporter activity in cultured SC cells and, further, that it is stimulated by hypertonic medium, which is consistent with the co-transporter function in volume regulation of these cells.

The effects of agents that alter TM cell volume on outflow facility of perfused human anterior chambers were previously examined and it was found that perfusing the chambers with hypertonic solutions, which cause a transient shrinkage of TM cells, resulted in a transient increase in outflow facility. The effect is expected to be transient because after the initial cell shrinkage, the co-transporter is activated to restore volume in the TM cells and, thus, the cell shrinkage is only transient. Similarly, perfusing the chambers with hypotonic solutions, which transiently swells TM cells, resulted in a transient decrease in outflow facility. Here again, the cells rapidly respond to altered volume, in this case by activating ion efflux pathways to decrease volume of the cells to normal. When the chambers were perfused with bumetamide, which inhibited the Na—K—Cl co-transporter and caused a sustained reduction of TM cell volume, a sustained elevation of outflow facility was observed. These findings suggest that volume of cells in the outflow pathway does influence outflow facility. A similar study by Gual et al. also found that conditions which increase cell volume cause a decrease in outflow facility (Gual, A. et al., Invest. Ophthalmol. Vis. Sci., 38: 2165-2171, 1997).

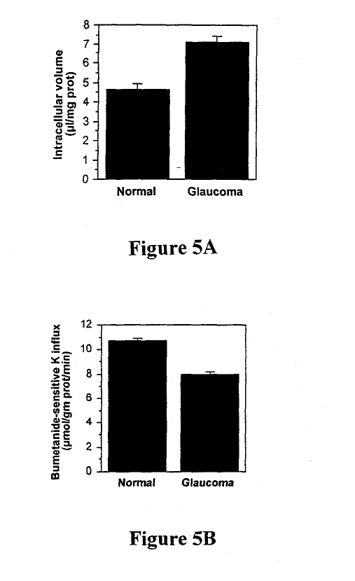

FIGS. 5A-B are bar graphs showing intracellular volume and co-transporter activity of normal human TM cells compared to glaucomatous human TM cells (isolated and cultured from trabeculectomy patients with primary open angle glaucoma). An important finding of these studies is that intracellular volume of glaucomatous human TM cells is significantly elevated, as shown in FIG. 5A. This indicates that the TM cell volume regulatory mechanisms may be aberrant in glaucoma.

Glaucomatous TM cells exhibited significantly reduced Na—K—Cl co-transporter activity, as shown in FIG. 5B, and reduced co-transporter protein expression. Moreover, unlike normal TM cells, the co-transporter of the glaucomatous cells appears to be insensitive to inhibition by cyclic AMP.

Despite the reduced co-transporter activity, the volume of both normal and glaucomatous TM cells is decreased by exposure of the cells to bumetamide. Thus, although the co-transporter activity is reduced, it still contributes to the maintenance of volume in both normal and glaucomatous cells because co-transporter inhibition by bumetamide decreases cell volume in both TM cell types. This indicates that reduction of cell volume by bumetamide may be of therapeutic value in increasing outflow facility of patients with glaucoma since our previous findings indicate that bumetamide is effective in increasing outflow facility of normal human anterior chambers. Despite this, the elevated resting volume in the glaucomatous TM cells, coupled with reduced co-transporter activity, suggests that defective co-transporter activity is not responsible for the elevated volume. Rather, it suggests that other volume-regulating pathways are defective, causing the volume to be increased which, in turn, would be expected to reduce activity of the co-transporter (since it is known that cell swelling reduces co-transporter activity). That other volume-regulating pathways account for the increased volume may be attributed to a defect in the volume-decreasing ion efflux pathways (.e.g., one that causes reduced K or Cl channel activities). Consistent with this possibility is the discovery that the Na—K—Cl co-transporter is sensitive to intracellular [Cl] levels; i.e., it is inhibited as intracellular [Cl] increases and stimulated as intracellular [Cl] decreases. This suggests that activity of the co-transporter is linked to Cl channel activity through intracellular [Cl] levels, as shown to be the case for a number of other cell types.

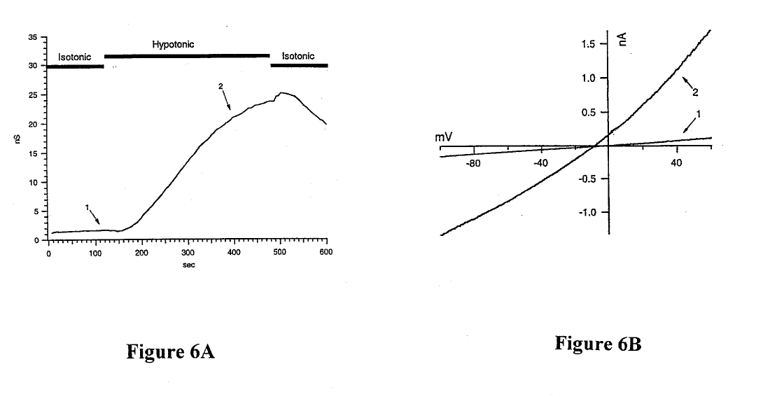

If Cl channels play an important role in regulating TM intracellular volume by mediating a regulatory volume decrease and also working in conjunction with the Na—K—Cl co-transporter to maintain steady state resting cell volume, then evidence of volume-sensitive Cl channels in TM cells should be observed. Thus, in electrophysiological studies, human TM cells were examined for the presence of swelling-activated Cl channels. Routine patch clamp methods were used and results were recorded from single TM cells in the whole cell patch clamp mode. FIG. 6A shows Cl conductance changes determined by whole cell recordings of a single TM cell subjected to isotonic (290 mOsm) and hypotonic (230 mOsm) bath medium. In this representative experiment, hypotonic medium (which swells the cells) caused an increase in Cl conductance of the cell. FIG. 6B shows the current/voltage (I/V) relationship recorded for a TM cell (representative experiment). The cells were subjected to an experimental protocol that included voltage ramps (+60 to −100 mV once every 5 seconds for 10 minutes; see legend). In this figure, line 1 is the I/V relationship for a cell exposed to isotonic bath medium and line 2 is the I/V relationship observed for the same TM cell in hypotonic bath medium. Arrows and numbers in FIG. 6A indicate time points at which I/V relationship shown in FIG. 6B were determined. The reversal potentials for these two I/V plots are the same and are consistent with the calculated Cl reversal potential of −5 mV, given the concentrations of Cl used in the bath and pipette (see legend). This indicates the swelling-activated conductance is a Cl conductance. In these experiments, Cl conductance in isotonic medium, consistent with a Cl conductance contributing to resting cell volume was also observed.

It has been observed that Cl channel blockers reduce activity of the Na—K—Cl co-transporter in human TM cells and that Na—K—Cl co-transporter activity is reduced when intracellular [Cl] is increased (Putney, L. K. et al., Amer. J. of Physiology: Cell Physiology, 277: C373-C383, 1999). These observations are consistent with the possibility that the Cl channel blockers cause an increase in intracellular [Cl] which in turn reduces co-transporter activity.

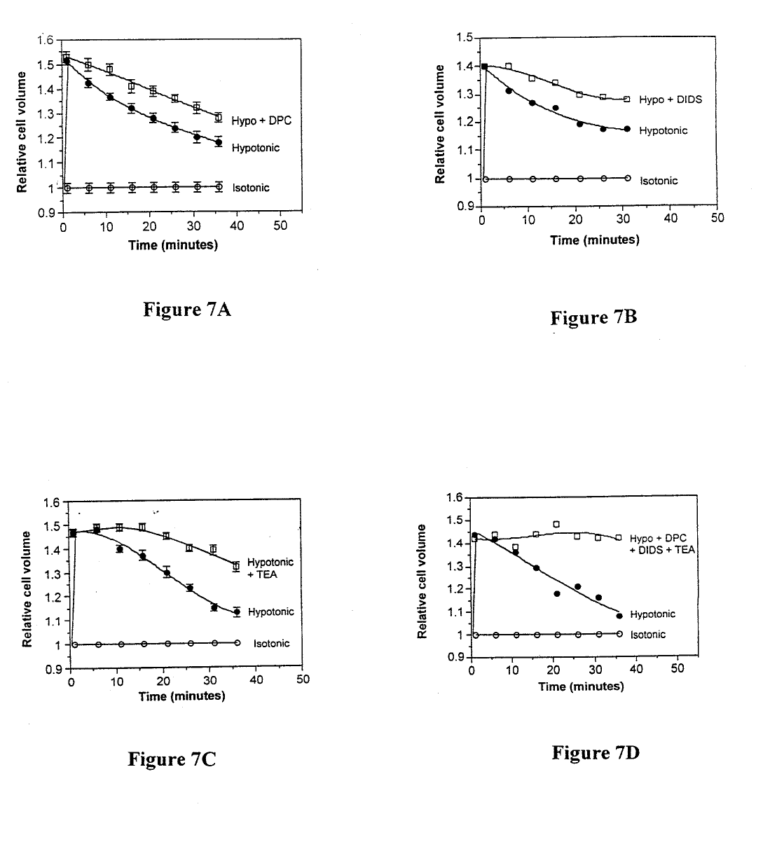

FIGS. 7A-D are line graphs that show the ability of TM cells to recover from hypotonic medium-induced cell swelling, i.e., to mediate a regulatory volume decrease (RVD). As predicted, exposing the cells to hypotonic media caused a rapid increase in cell volume followed by a return of volume back toward normal levels as shown in FIG. 7A. Exposure of the cells to hypotonic medium containing DPC, an agent that blocks Cl channels, attenuated the RVD. Exposure of the cells to DIDS, another agent that blocks Cl channels, reduced the RVD as shown in FIG. 7B. Also tested was the effect of blocking K channels on the RVD. In cells that use volume-sensitive Cl channels to mediate the RVD, generally, K channels operate in parallel to mediate K efflux along with the Cl efflux. Thus, even if Cl channels are activated by cell swelling, preventing K efflux via the K channels will attenuate the RVD (Cl efflux will be attenuated as the cells depolarize). Consistent with this, when cells were exposed to TEA to block K channels, the RVD was also diminished as shown in FIG. 7C. Exposing the TM cells to hypotonic medium containing DPC, DIDS and TEA in combination caused a complete inhibition of the RVD as shown in FIG. 7D. These findings suggest that Cl channels and K channels are important in TM cell volume regulation.

In further experiments looking at the role of K—Cl co-transport in volume regulation of TM cells, it was found that furosemide (1 mM, a concentration that blocks K—Cl co-transport) had no effect on the RVD of TM cells, nor did the cells appear to have furosemide-sensitive K influx.

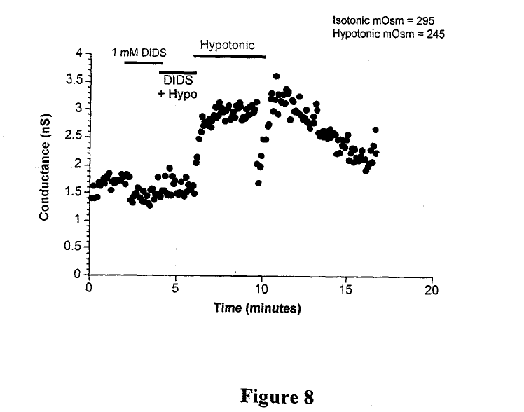

The presence of swelling-activated Cl channels in normal human TM cells are also shown in FIG. 8 using electrophysiological methods. The ion conductances of human TM cells exposed to hypotonic media were evaluated. Switching the cells from isotonic to hypotonic medium caused a rapid increase in conductance. Swelling activated conductances that have been described in other cells include K channel-mediated conductances and also Cl channel-mediated conductances. To determine the predominant channel that mediates the increased conductance, the reversal potential of the current/voltage relationship can be determined. A reversal potential close to the reversal potential calculated for activation of a Cl channel indicates that the swelling-activated conductance is mediated primarily by opening of Cl channels. Similarly, a reversal potential close to that calculated for activation of a K channel indicates that the swelling-induced conductance is primarily due to K channels. In experiments, the observed swelling-activated conductance had a reversal potential very close to that calculated for Cl. This indicates that the activated conductance is primarily mediated by opening of a Cl channel. Consistent with this, Cl channel blockers DIDS and NPPB abolished the cell swelling (hypotonic medium)-induced increase in TM cell ion conductance.

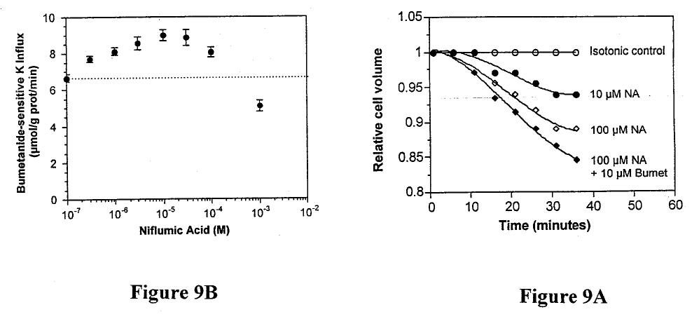

In previous studies of the effects of Cl and of Cl channel blockers on TM cell Na—K—Cl co-transporter activity, surprisingly, one of the putative Cl channel blockers, niflumic acid, did not inhibit co-transporter activity; but, instead, it actually stimulated activity of the co-transporter. This was a concentration-dependent effect and occurred at concentrations below 1 mM. As shown in FIG. 9A, concentrations of niflumic acid from 0.3 μM to 100 μM were found to significantly stimulate Na-K-Cl co-transporter activity. This is consistent with previous findings that although niflumic acid inhibits Cl channels in a number of cell types, it appears to activate Cl channels in retinal pigment epithelial cells. Niflumic acid (100 μM) significantly reduced intracellular [Cl] of the TM cells. This suggests that niflumic acid could activate TM or SC cell Cl channels to decrease intracellular [Cl] which in turn activates the Na—K—Cl co-transporter. In addition, niflumic acid caused a significant decrease in TM cell volume. Both the cell shrinkage and the decreased intracellular [Cl] induced by niflumic acid would be expected to stimulate co-transporter activity.

The effect of niflumic acid on TM cell volume is shown in FIG. 6B. Both 10 μM and 100 μM niflumic acid decreased TM cell volume in isotonic medium (i.e., resting volume, not perturbed by anisosmotic media). Further, when the cells were placed in isotonic medium containing both 100 μM niflumic acid and 10 μM bumetamide, the cell volume decreased to an even greater degree than with either niflumic acid alone or bumetamide alone. That is, activation of the channels would decrease intracellular [Cl] and shrink the cells. The co-transporter would be stimulated by the decreased intracellular [Cl] and shrunk cells to work against the Cl efflux and maintain cell volume. However, when the co-transporter is blocked, Cl efflux is unopposed and the cell shrinkage continues unchecked by the co-transporter. It appears that the two compounds may have a synergistic effect on cell volume regulation.

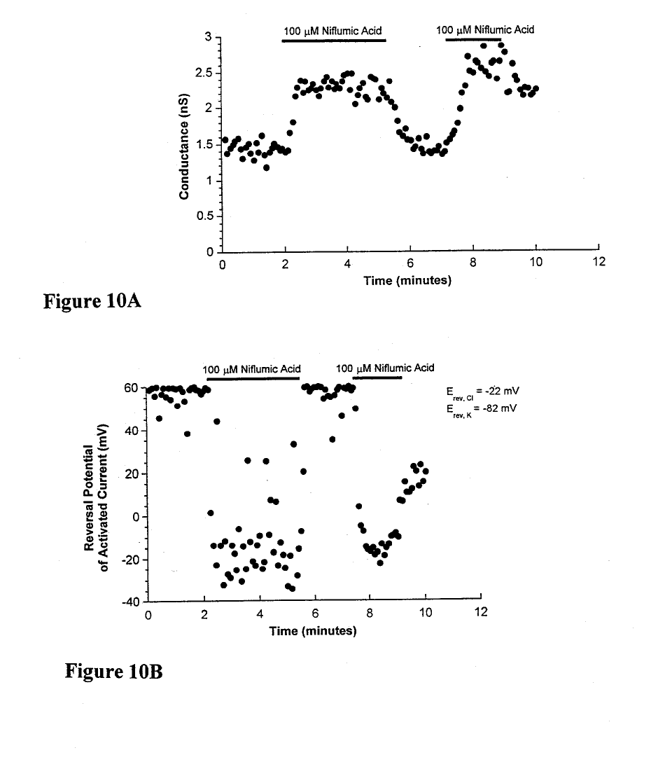

FIGS. 10A-B show the results of electrophysiology experiments to evaluate the possibility that the observed TM cell shrinkage caused by niflumic acid is due to activation of a Cl channel. In these patch clamp whole cell recording experiments, exposure of normal human TM cells to 100 μM niflumic acid does activate an ion conductance in the cells. The reversal potential of the niflumic acid-activated conductance is quite close to the calculated reversal potential for Cl under the given experimental conditions. This suggests that a Cl channel does mediate the niflumic acid-activated conductance in these cells.

Further studies conducted with agents known to block Cl and K channels attenuate the swelling-activated regulatory volume decrease in TM cells and niflumic acid reduces both cell volume and intracellular [Cl] of TM cells. In these studies, human TM cells were cultured from donor eyes and used between passages three and eight. Routine whole cell patch clamp methods were used to assess conductances of TM cells in isotonic (290 mOsm) and hypotonic (230 mOsm) media, with or without NPPB, DIDS, TEA or niflumic acid. Conductances were determined from current/voltage plots, generated using a ramp protocol. The result of the conductance studies shows that exposure of human TM cells to hypotonic medium caused a large increase in ion conductance within one minute. The reversal potential observed in the presence of hypotonic medium was close to the calculated reversal potential for Cl. Switching to an external medium containing reduced Cl shifted the reversal potential toward that calculated for the low Cl medium. Treating the TM cells with the Cl channel blocker NPPB (100 μM) abolished the swelling-activated conductance in a manner reversed by subsequent washout of NPPB. DIDS (1 mM) also blocked the swelling-activated conductance. Exposing the TM cells to niflumic acid (100 μM) also caused a rapid increase in conductance. The niflumic acid-activated conductance was abolished in K-free medium but not in Cl-free medium. Finally, treatment of the cells with the K channel blocker TEA (1 mM) also abolished the niflumic acid-activated TM cell conductance.

These results indicate that hypotonic medium-induced cell swelling of human TM cells activates an ion conductance mediated primarily by Cl channels. Treatment of human TM cells with niflumic acid appears to activate a K conductance, suggesting that niflumic acid may also reduce TM cell volume by a mechanism involving K channel activation.

It would seem that Na—K—Cl co-transport, K channels and Cl channels are essential components to TM and SC cell volume regulation. Cell volume regulation requires both volume-increasing ion flux pathways (the Na—K—Cl co-transporter) and volume-decreasing ion flux pathways (K and Cl channels) and that intracellular volume is determined by the combined activities of these opposing pathways. The finding that glaucomatous TM cell volume is elevated compared to normal TM cells, coupled with the observed decrease in co-transporter activity in these cells, points to a possible defect in responsiveness of volume-sensitive K and/or Cl channels in glaucomatous TM and/or SC cells.

It is preferable that compounds to be administered to the eye topically in the practice of this invention not only activate K and/or Cl channels, but also be sufficiently lipophilic to penetrate the corneal membrane. The lipophilicity of a compound is expressed in terms of an octanol:water coefficient, determined by the standard technique of radiolabelling the compound and introducing a small amount into equal volumes of octanol and tris buffer (50 mM, pH 7.4). Generally the lipophilicity (log P′) is expressed as the logarithm of the partition coefficient in n-octanol/phosphate buffer, pH 7.4 using the well known shake-flask method as described by Cloux, et al., J. Pharm. Belg., 43:141-151, 1973, which is incorporated herein by reference in its entirety. The coefficient of lipophilicity (log P′) of the compounds useful for topical application to decrease intra-ocular pressure is preferably at least 0.005, and more preferably at least 0.01.

The lipophilicity of the aqueous humor outflow-increasing compounds of this invention can also be determined using a reversed phase, high performance liquid chromatograph (RP-HPLC) system for determination of the log P′ of the drug as described in B. Masereel, et al., J. Pharm. Pharmacol. 44:589-593, 1992, which is incorporated herein by reference in its entirety. Briefly, a reversed phase column (RP-18) is equilibrated with n-propanol/phosphate buffer, pH 7.4 at a ratio of 30:70). Compounds to be tested are dissolved and eluted with the same solution. A series of standards with a wide range of lipophilicity, as determined by the shake-flask method, is run and a calibration curve is established for each session. KNO₃ is injected to determine the void volume and log k′=log(tr−to)/to is determined, wherein tr is the drug retention time and to is the retention time of NO₃⁻. Calibration curves are calculated using log P′ and log k′ values. Log P′ values of other compounds are obtained by interpolation of the standard curves.

Among the preferred outflow-increasing compounds of this invention are lipophilic derivatives of niflumic acid and flufenamic acid, and biologically compatible salts thereof, which, in proper doses, are potent activators of the K and/or Cl channels. These compounds combine a high degree of lipophilicity and biological activity. The outflow-increasing compounds disclosed herein, which activate K and/or Cl channels, can be administered either topically or by microinjection into the eye in, near, or about the TM and/or SC cells. For topical administration, the compound can be dissolved in a pharmaceutically acceptable carrier substance, e.g., physiological saline. Additional pharmaceutically acceptable carrier substances can readily be supplied by one skilled in the art. For compounds having limited water solubility, the liquid carrier medium can contain an organic solvent, for example, 3% methyl cellulose. Methyl cellulose provides, by its high viscosity, increased contact time between the compound and the surface of the eye, and may, therefore, increase corneal penetration. Corneal penetration can also be increased by administering the compound mixed with an agent that slightly disrupts the corneal membrane, for example 0.025% benzalkonium chloride, which also serves as a bacteriostatic preservative in various commercial formulations. Corneal penetration may also be increased by delivering a suspension of liposomes that incorporate the therapeutic compound, as described by Davies et al., (“Advanced Corneal Delivery Systems: Liposomes” in Opthalmic Drug Delivery Systems, A. K. Mitra, Ed., Vol. 58, pages 289306 in the series Drugs and the Pharmaceutical Sciences, 1993, Marcel Dekker, Inc., New York), which is incorporated herein by reference. The outflow-increasing compound may be administered periodically (for example, one time per week to ten times per day). Administration may be by applying drops of the compound in solution using an eye dropper, such that an effective amount of the compound is delivered through the cornea to the trabecular meshwork and/or Canal of Schlemm endothelial cells. Administration may also be by a sustained-release formulation, such as a liposome, or via an ocular insert designed to enhance the dwelling time of the compound in the tear film and improve patient compliance with therapy, such as those described by R. Bawa, in A. K. Mitra, Ed., supra, Chapter 11, pages 223-260.

The “effective amount” of the compound to be delivered in one administration will depend on individual patient characteristics, e.g. the severity of the disease, as well as the characteristics of the administered compound, such as its lipophilicity and biological activity in stimulating the K and/or Cl channels or inhibiting the Na—K—Cl co-transporter. Generally, an “effective amount” is that amount necessary to substantially activate the K and/or Cl channel mechanism, inhibit the Na—K—Cl co-transporter mechanism, or establish homeostasis of the aqueous fluid in the eye as indicated by the intra-ocular pressure. Intra-ocular pressure reflects the balance between the production and outflow of aqueous humor, and the normal range is 2.09″0.33 kPa (15.8″2.5 mmHg) as measured by applanation tonometry (by planating the corneal surface) (Harrison's Principles of Internal Medicine, 13th Ed., Isselbacher et al., Ed., McGraw Hill, Inc., New York, p. 105). Systemic absorption of the drug can be minimized by digital compression of the inner canthus of the eye during and for a short time following its instillation into the eye.

Direct microinjection of the solubilized compound to a site near the TM and/or SC cells offers the advantage of concentrating the compound in the location where it is needed, while avoiding the possibility of side effects resulting from generalized exposure of the eye to the compound. Microinjection may also provide the advantage of permitting infrequent periodic administration, for example every few weeks, months, or even years, in contrast to the more frequent administrations required in the case of topical administration. Also, direct microinjection may promote the washing out of the trabecular meshwork or Schlemm's canal of extracellular material interfering with fluid outflow. Preferably microinjection is administered via subconjunctival injection, most preferably into the superior aspect of the globe at the 12:00 o'clock position, from which point the drug reaches the intra-ocular space by diffusing passively across the scleral fibers, which offer essentially no barrier to penetration. Dosage for microinjection, like that for topical administration, varies with the above-mentioned parameters.

The following examples illustrate the manner in which the invention can be practiced. Using these examples, other compounds may be screened for their utility in increasing aqueous humor outflow. It is understood, however, that the examples are for the purpose of illustration and the invention is not to be regarded as limited to any of the specific materials or conditions therein.

### EXAMPLE 1

A. Cell Culture.

Bovine trabecular meshwork (TM) cells (Department of Ophthalmology, Lions of Illinois Eye Research Institute, Chicago, Ill.) and human trabecular meshwork isolated by methods based on those of Polansky et al., (Invest. Ophthalmol. Vis. Sci., 18:1043-1049, 1979). Briefly, for isolation of bovine TM cells, eyes from healthy, freshly slaughtered young cows were enucleated. The TM was surgically excised, taking care not to include surrounding tissues. Explants were cut into small pieces (˜1 mm³), put in collagen-coated 175 cm² tissue culture flasks without medium for 1 minute until adhering, then growth medium was added to the flask. The media used was Eagle's minimal essential medium (MEM) supplemented with 10% fetal bovine serum (Hyclone Laboratories, Logan, Utah), essential and non-essential amino acids, glutamine, and penicillin/streptomycin. The explants were maintained in a humidified CO₂ incubator at 37° C. and 5% CO₂. When cells growing out of the explant reached confluence, they were trypsinized and subcultured. Cultures that appeared to contain non-trabecular meshwork cells were discarded. Cultures were maintained by refeeding every 2 days and splitting weekly.

Similar techniques were used in isolation of human TM cells, except that human TM derived from three sources: 1) research donor eyes (presumed to be normal) from the eye bank of the University of California, Davis Medical Center; 2) otherwise healthy eyes enucleated because of life-threatening malignancies in the posterior pole (e.g., retinoblastoma or choroidal melanoma); and 3) trabeculectomy specimens. At the time of trabeculectomy surgery, the surgeon created a partial thickness scleral flap, unroofing the TM at the surgical limbus. A small piece (0.1 to 2 mm³) was then excised to create the surgical fistula.

Both types of TM cells were maintained in collagen-coated tissue culture flasks and were used between passages 8 and 12 for bovine and between passages 3 and 8 for human. For experiments, cells were removed from the flasks by brief typsinization and were subcultured onto 24 well plates coated with collagen Type I (Collaborative Research, Inc., Bedford, Mass.) for radioisotopic transport and cell volume experiments or onto collagen-coated tissue culture filter inserts (BIOCOAT™, 13 mm diameter, 0.45 μm pore size (Collaborative Research Inc.). Cells were used 5-7 days later as confluent monolayers and growth medium was replaced every 2 days.

Canal of Schlemm endothelial cells were obtained by Dr. Dan Stamer, as described in Stamer, W. D., et al., Investigative Ophthalmology and Visual Science, 39:1804-1812, 1998. Dr. Stamer's method has been to isolate cells from human cadaveric eye tissue obtained from the National Disease Research Interchange (NDRI, Philadelphia, Pa.) within 48 hours of death for whole eyes stored in moist chambers and 96 hours of death for nontransplantable corneal anterior segments stored in solution (OPTISOL, Chiron Vision, Clairmont, Calif.). Donor rims were made available by Dr. Mark Mannis, corneal transplant surgeon at U.C. Davis Medical Center. These donor rims contain TM and SC cells after the central cornea is punched out and used for transplant). These donor rims will work well for isolation of SC cells using Dr. Stamer's methods. By this approach, the anterior chamber of the eye (or donor rim) is cut into eight wedge-shaped pieces. Using a dissecting microscope, a gelatin-coated suture (6-0 sterile nylon monofilament, Wilson Ophthalmic, Mustang, Okla.) is gently inserted into the lumen of Schlemm's Canal and advanced completely through the canal in the tissue section. The cannulated pieces are then placed into culture, using Dulbecco's modified Eagle's medium (DMEM) with 10% fetal bovine serum (FBS) and penicillin/streptomycin. The pieces are then maintained in a tissue culture incubator (7% CO₂) for at least three weeks. Sutures are then removed from the SC lumens and cells (attached to the gelatin-coated sutures) seeded onto culture dishes. The SC cells are then maintained in culture in the DMEM/10% FBS medium. Confluent cultures are subcultured by gentle typsinization.

B. Transport Measurements.

Agents known to increase aqueous outflow should inhibit activity of the co-transporter and/or activate K or Cl channels and agents which decrease aqueous outflow should activate activity of the co-transporter and/or inhibit the K or Cl channels. Further these agents should alter Na⁺—K⁺—2Cl⁻ co-transport, K channel and/or Cl channel with a potency similar to that observed for their actions on trabecular meshwork function.

Na—K—Cl co-transport was measured as ouabain-insensitive, bumetamide-sensitive potassium influx, using ⁸⁶Rb as a tracer for potassium. Details of this method have been published previously (O'Donnell, M. E., supra, 1989). Briefly, bovine or human TM cell monolayers on 24 well plates were equilibrated for 10 minutes at 37° C. in a Hepes-buffered minimal essential medium (MEM) containing (in mM): 144 Na, 147 Cl, 5.8 K, 1.2 Ca, 4.2 HCO₃, 0.4 HPO₄, 0.4 H₂PO₄, 0.4 SO₄, 5.6 glucose and 20 Hepes. The cells were then preincubated and assayed for 5 minutes each with Hepes-buffered MEM containing 1 or 0 mM ouabain (Boehringer-Mannheim Biochemicals, Indianapolis, Ind.), 10 or 0 μM bumetamide (Hoffman-LaRoche, Nutley, N.J.) and either 145 or 0 mM Na and Cl (Na isosmotically replaced by choline, Cl isosmotically replaced by gluconate). The assay medium also contained ⁸⁶Rb (1 μCi/ml) (Dupont New England Nuclear, Boston, Mass.). The assay was terminated by rinsing the wells with ice-cold isotonic MgCl₂, then extracting the contents with 0.2% sodium dodecyl sulfate (SDS), and determining the amount of radioactive contents by liquid scintillation. Osmolarities of all preincubation and assay media were verified by osmometry (Model 3W2, Advanced Instruments, Norwood, Mass.).

C. Measurements of Cell Volume.

The intracellular volume of human and bovine TM cells was evaluated by two methods: 1) radioisotopic evaluation of TM monolayer intracellular water space using ¹⁴C-urea and ¹⁴C-sucrose as markers of total and extracellular space, respectively; and 2) electronic cell sizing of suspended TM cells, using a COULTER COUNTER™ assay (Coulter Electronics, Ltd., Hialeah, Fla.). Details of these methods have been described previously by O'Donnell (O'Donnell, M. E., supra, 1993) the entirety of which is incorporated herein by reference.

By the first method, cell monolayers were preequilibrated for 30 minutes in Hepes MEM at 37° C. in an air atmosphere, then incubated for 20 minutes in Hepes MEM containing 0 or 10 μM bumetamide, ethacrynic acid, or other agents to be tested, and finally incubated for 10 minutes in the same medium containing either ¹⁴C-urea or ¹⁴C-sucrose (both at 1 μCi/ml). Monolayers were then rinsed with isotonic ice-cold MgCl₂ and radioactivity of SDS extracts determined by liquid scintillation. Specific activities (counts per minute/ml) of ¹⁴C-urea and ¹⁴C-sucrose in the assay medium were determined and used to calculate water space associated with trapped radioactivity (expressed as μl/mg protein. Intracellular volume was calculated as the difference between the water space determined for ¹⁴C-urea (a marker for intracellular plus trapped extracellular space) and ¹⁴C-sucrose (a marker for trapped extracellular space).

By the second method, TM cells were trypsinized briefly in Ca-free medium, then rinsed with medium containing trypsin inhibitor and suspended in Hepes MEM. Mean cell volumes were then analyzed by electronic cell sizing on a COULTER COUNTER™ radioassay (Model ZM) with channelizer (C256), using at least 50,000 cells per data point and an orifice diameter of 140 μm. Aliquots of suspended cells were diluted into Hepes MEM containing the tonicity and/or agents to be evaluated. Mean cell volumes of each suspension aliquot were followed over time, starting at 1 minute after addition of cells to the assay media. Cell suspensions were maintained at 37° C. throughout the assay period. Absolute volumes (picoliters/cell) were calculated from distribution curves of cell diameter, using a standard curve generated by polystyrene latex beads of known diameter.

D. Electrophysiological Evaluation of Cl Channel Conductances in Human TM and SC Cells.

Human TM and SC cells grown to subconfluence are gently trypsinized on the day of the experiment and immediately exposed to trypsin-neutralizing solution and then allowed to settle onto standard square (22×22-mm) glass coverslips for 30 minutes before use. Routine patch clamp methods were used as described previously Barakat, A. I., et al., Circulation Research, 85: 820-828, 1999; Hamill, O. P. et al., Pflügers Archives, 391: 85-100, 1981; Mauro, T. et al., J of Invest. Dermatology, 105: 203-208, 1995; Pappone, P. A. et al., J. of Physiology, 306:377-410, 1980; Pappone, P. A. et al., J of Gen. Physiology, 106: 231-258, 1995; Pappone, P. A. et al., Amer. J. of Physiology: Cell Physiology, 264: C1014-C1019, 1993; Wilson, S. M. et al., J. of Gen. Physiology, 113: 125-138, 1999. Using the whole cell patch clamp mode, conductances were assessed for isotonic and hypotonic media. By assessing current/voltage (I/V) relationships and reversal potentials for the TM and SC cells while using specific concentrations of Cl in the pipette and bath media, the nature of the conductances observed can be determined.

Whole cell currents were measured at room temperature in a Warner recording/perfusion chamber (Warner Instrument Corp.) using standard whole-cell patch clamp techniques as described previously, Lucero, M. T. et al., J. of Gen. Physiology, 95:523-544, 1999. Thick-walled borosilicate capillaries (Sutter Instruments, Inc., Novato, Calif.) were used to manufacture pipettes with resistances of 3 to 5 MW. The voltage offset between the patch pipette and the bath solution were nulled immediately before patch formation. An agar bridge containing 1 mM KCl was used to ground the bath solution. Voltages were recorded from a patch-clamp amplifier (AXOPATCH 200A, Axon Instruments). Data was collected on a Macintosh personal computer using Pulse+Pulsefit software (HEKA Elektronik). Whole-cell currents were recorded from single (sub confluent) cells with membrane capacitances nulled through the patch-clamp amplifier. The voltage ramp protocol included a voltage step from the holding potential to +60 mV for 40 ms followed by a ramp to −100 mV over a period of 400 ms followed by a return to the holding potential. During the protocol, voltage ramps were conducted once every 5 seconds for 10 minutes, yielding 120 traces. Conductances were computed by taking the derivative of a least-squares straight line fit to the I-V data plots using Igor Pro Software (Wavemetrics, Inc.). In cases in which the I-V plots are not linear over the entire voltage ramp (as in FIGS. 6AB), computation of the whole-cell conductance were restricted to a linear range, typically 0 to +60 mV.

Solutions: The patch pipette fill solution will consist of (in mM) 100 K aspartate, 35 KCl, 10 K2-EGTA, 1 CaCl₂, 10 MOPS, 4 ATP and 4 MgCl₂, with the pH adjusted to 7.2 with NaOH. ATP/MgCl₂ solution (pH 7.2 with NaOH) will be added to the fill solution from a frozen stock at the beginning of each day. Cells will be continuously perfused with a bath solution which is based on Hepes-buffered minimal essential medium (MEM), i.e., containing (in mM): 143 Na, 136 Cl, 5.8 K, 1.2 Ca, 4.2 HCO₃, 0.33 HPO₄, 0.4 H₂PO₄, 0.81 Mg, 0.81 SO₄, 5.6 glucose and 20 Hepes, pH 7.4. For experiments testing the effects of hypotonicity, osmolarity will be varied by removal of NaCl (hypotonic media) or addition of NaCl or dextrose (for hypertonic media). The osmolarity of all media will be determined by osmometry. This bath medium will be varied in other ways as required by each experiment. Thus, for example, in some experiments, NaCl will be replaced by the sodium salt of another anion (e.g. NaBr or NaI). Also, experiments examining Cl conductances may also be made in a potassium-free Hepes-buffered solution to minimize currents through K channels.

E. Statistical Analysis.

Experimental results were analyzed for statistical significance using Student's t test or Analysis of Variance, with a p value <0.05 used as the criterion for significance.

### EXAMPLE 2

Experiments using protocol described in Example 1, Section B.

Evaluation of K influx in human trabecular meshwork cells reveals the presence of a robust Na—K—Cl co-transport system. As shown in FIG. 2A, normal human TM cell monolayers were cultured on multiwell plates. The cells were pretreated with Hepes-buffered minimal essential medium (MEM) ±10 μM bumetamide ±1 mM ouabain for 5 min, then assayed for 5 min in the same media plus ⁸⁶Rb. Bumetamide-sensitive K influx can be observed as the difference between the dark gray bar (control) and the black bar (bumetamide); or as the difference between the white bar (ouabain) and the light gray bar (ouabain+bumetamide). Data are means ±SEM, n=12.

Evaluation of Na—K—Cl co-transport activity in human Schlemm's Canal endothelial cells as shown in FIG. 4. Human Schlemm's Canal endothelial cells were provided by Dr. Dan Stamer. The cells were isolated and cultured by the Stamer lab as described previously. At the O'Donnell laboratory, the cells were subcultured and set up on collagen-coated 24 well cluster plates for assay. Na—K—Cl co-transport was assessed as bumetamide-sensitive K influx, using ⁸⁶Rb as a tracer for K as described previously. For this assay, cells were preincubated for 5 minutes in either an isotonic (300 mOsm) or hypertonic (400 mOsm) Hepes-buffered medium containing 0 or 10 μM bumetamide. The cells were then assayed for 10 minutes in either isotonic or hypertonic assay media with 0 or 10 μM bumetamide and also 1 μCi/ml ⁸⁶Rb. Data represent means ±SEM of a single experiment with quadruplicate replicates.

TM cell Na—K—Cl co-transporter activity is reduced in glaucomatous cells as shown in FIG. 5B. Na—K—Cl co-transport activity of glaucomatous human TM cells was assessed as ouabain-insensitive, bumetamide-sensitive K influx, using ⁸⁶Rb as a tracer for K, as described previously. TM cells were preincubated for 5 minutes in a Hepes-buffered MEM containing 0 or 10 μM bumetamide in the presence of 1 mM ouabain, followed by a 5 minute assay in media of identical composition except that they also contained ⁸⁶Rb. Co-transport activity was significantly decreased in glaucomatous TM cells compared to activity in normal TM cells. Data are mean values ±SEM in seven normal TM cultures (donor age range, 38-68 years, n=12 in each cell culture) and in seven glaucomatous cultures (donor age range 41-75 years, n=7 in each cell culture).

Niflumic acid effects on human TM cell Na—K—Cl co-transporter activity are shown in FIG. 9A. Human TM cells were equilibrated in Hepes-buffered MEM for 30 minutes and then pretreated for 5 minutes in media containing 1 mM ouabain, 10 or 0 μM bumetamide, and 0.3 μM to 1 mM niflumic acid (NA). Cells were then assayed for bumetamide-sensitive K influx for 5 minutes in media identical to the pretreatment media but also containing ⁸⁶Rb (1 μCi/ml). The K influx value for 0 mM NA control was not significantly different from that at 0.1 μM. Dashed line indicates control level of Na—K—Cl co-transport. Data are means ±SEM of at least 4 determinations from 3 experiments.

### EXAMPLE 3

Experiments using the protocol described in Example 1, Section C.

The Na—K—Cl co-transport inhibitor bumetamide abolishes the TM cell regulatory volume increase as shown in FIG. 3A. Confluent bovine trabecular meshwork cell monolayers were exposed briefly to trypsin (0.1%) and suspended in isotonic Hepes-buffered minimal MEM. Cells were then diluted into isotonic medium (300 mOsm) or hypertonic medium (400 mOsm, by addition of NaCl). Mean cell volume was then determined over the time course shown by electronic cell sizing with a COULTER COUNTER™. Data are means ±SEM, n=4.

FIG. 3B shows the Na—K—Cl co-transporter of TM cells is stimulated by hypertonic shrinkage. Bovine TM cell monolayers, attached to multiwell plates, were assayed for bumetamide-sensitive K influx in media of varying tonicity (altered by addition of NaCl). Data are means ±SEM, n=4. The human TM cell Na—K—Cl co-transporter is also stimulated by hypertonic medium with a 54% increase in activity observed with 400 mOsm medium compared to isotonic control medium (data not shown).

TM cell volume is elevated in glaucomatous cells as shown in FIG. 5A. Intracellular volume of normal and glaucomatous human TM cells was assessed radioisotopically, as described in Example 1, Section C. TM cell monolayers were assayed for 10 minutes in isotonic Hepes-buffered MEM containing ¹⁴C-urea or ¹⁴C-sucrose. Intracellular water was calculated as the difference between water space determined for ¹⁴C-urea (a marker for total water space, i.e., intracellular plus trapped extracellular space) and ¹⁴C-sucrose (a marker for trapped extracellular space). Intracellular volume of glaucomatous TM cells was significantly increased compared to normal TM cells. Data are means ±SEM in three normal cultures (donor ages 48,55 and 60 years, n=12 in each culture) and in three glaucomatous cultures (donor ages 48, 75 and 75 years, n=12 in each culture).

FIGS. 7A-D show the reduction of the regulatory volume decrease (RVD) in human TM cells by Cl and K channel blockers. In FIG. 7A, cells were exposed to isotonic medium (300 mOsm) or to hypotonic medium (200 mOsm)±1 mM DPC (diphenylamine carboxylate). Cell volume was assessed by COULTER COUNTER™ electronic cell sizing over the time course shown, using TM cells in suspension. Cells in hypotonic medium. alone showed a regulatory volume decrease (RVD), which was attenuated by DPC. Data represent means of two experiments, error values shown are SEM, n=4 (two separate RVD runs per experiment).

FIG. 7B shows human TM cells exposed to isotonic or hypotonic medium ±1 mM DIDS (4,4′-diisothiocyanostilbene-2,2′-disulfonic acid). Note that DIDS attenuated the RVD.

FIG. 7C shows human TM cells treated with isotonic or hypotonic medium ±the K channel blocker TEA (tetraethylammonium, 1 mM). Data represent means of two experiments, error values shown are SEM, n=4 (two separate RVD runs per experiment).

FIG. 7D shows human TM cell suspensions treated with isotonic or hypotonic medium ±DIDS+DPC+TEA (all at 1 mM). The effects of these channels blockers are additive and abolished the regulatory volume decrease.

FIG. 9B shows the additive volume-reducing effects of bumetamide and niflumic acid. Human TM cells were exposed to isotonic medium ±niflumic acid (10 or 100 μM)±10 μM bumetamide. Cell volume was assessed by COULTER COUNTER™ electronic cell sizing over the time course shown, using TM cells in suspension. Note that niflumic acid (NA) alone decreases volume of the TM cells and that exposing the cells to NA plus bumetamide causes a further decrease in TM cell volume. Data represent means of two experiments (all conditions shown tested in both experiments).

### EXAMPLE 4

Experiments using the protocol described in Example 1, Section D.

FIGS. 6A-B show Cl conductance changes induced by hypotonic medium and current voltage relationship for TM cells in isotonic and hypotonic media, respectively. Line 1 is for TM cell exposed to isotonic bath medium. Line 2 is for TM cell exposed to hypotonic bath medium. Arrows and numbers in FIG. 6A indicate time points at which current voltage relationships shown in FIG. 6B were determined. For these experiments, borosilicate pipettes used had resistances of 3 to 5 MW. The voltage offset between the patch pipette and the bath solution was nulled immediately prior to seal formation. Voltages were recorded from a patch-clamp amplifier (AXOPATCH 200A, Axons Instruments) in current clamp mode (I=0). Data were collected on a Macintosh personal computer using Pulse+Pulsefit software (HEKA Elektronik). During the experimental protocol, voltage ramps (400 msec duration) were conducted once every 5 seconds for 10 min yielding 120 traces. Conductances were computed by taking the derivative of a least-squares straight line fit to the I-V data plots using Igor Pro software (Wavemetrics, Inc.). For the figures shown, [Cl] in the bath medium was 45.3 mM and [Cl] in the pipette was 37 mM. The calculated Cl reversal potential is −5 mM, in agreement with the data shown, indicating that the conductance is mediated by a Cl channel.

FIG. 8 shows the swelling-activated ion conductance in human TM cells and inhibition by Cl channel inhibitors. Whole cell recordings were made of a single TM cell subjected to isotonic (295 mOsm) or hypotonic (245 mOsm) bath medium in the presence or absence of the Cl channel inhibitor DIDS (1 mM) in the bath medium. FIG. 8 also shows the conductance changes induced by hypotonic medium. Current/voltage (I/V) plots were generated over the time course shown using the patch-clamp ramp protocol described below. Conductances were determined from the I/V plots, using the slopes for voltages between 0 to 60 mV. Cells were first exposed to isotonic medium (first 2 min), then to isotonic bath medium containing DIDS. The bath medium was subsequently switched to hypotonic medium containing DIDS and finally, to hypotonic medium without DIDS before switching back to isotonic medium without DIDS, as indicated in the figure (medium is isotonic without DIDS for the areas without bars (before 3 min and after 10 min on the figure). Note that conductance does not increase in hypotonic medium when DIDS is present but as soon as the cells are exposed to DIDS-free hypotonic medium, conductance increases. Return to isotonic medium decreases the conductance back toward pre-hypotonic levels. The line of data points that lie below the hypotonic conductance values at the end of the hypotonic period are due to an artifact related to the external solution delivery system that was used in this experiment, caused by a small amount of DIDS being introduced at the start of the second isotonic period (but rapidly washed out).

Not shown is a figure of the reversal potentials determined from the same I/V plots used to calculate conductances shown in the FIG. 8. For the given experimental conditions, the calculated reversal potentials for Cl and K are −28 mV and −77 mV, respectively. Note that the observed reversal potential in the presence of hypotonic medium is very close to the Cl reversal potential, indicating that hypotonic medium activates a Cl channel to produce the increase in conductance (as opposed to activating a K channel, which should give a reversal potential close to −77 mV rather than −28 mV). For these experiments, borosilicate pipettes used had resistances of 3 to 5 MΩ. The voltage offset between the patch pipette and the bath solution was nulled immediately prior to seal formation. Voltages were recorded from a patch-clamp amplifier (AXOPATCH 200A, Axons Instruments) in current clamp mode (I=0). Data were collected on a Macintosh personal computer using Pulse+Pulsefit software (HEKA Elektronik). During the experimental protocol, voltage ramps were conducted once every seconds for 10 min yielding 120 traces. Conductances were computed by taking the derivative of a least-squares straight line fit to the I-V data plots using Igor Pro software (Wavemetrics, Inc.). For this experiment, [Cl] and [K] in the bath medium were 107.9 mM and 5.8 mM, respectively, while [Cl] and [K] in the pipette were 35.8 mM and 125.4 mM, respectively. Similar results were obtained in seven experiments in which the Cl channel inhibitor NPPB (100 μM) was used, i.e., the hypotonic medium-activated conductance was blocked by NPPB.

FIGS. 10A-B show the effects of niflumic acid on human TM cells and is evidence for activation of Cl channels. Whole cell recordings were made of a single TM cell subjected to bath medium containing 0 or 100 μM niflumic acid.

FIG. 10A shows Cl conductance changes induced by niflumic acid. Conductances were determined as described above for FIG. 8. Cells were exposed to bath isotonic bath medium with or without niflumic acid and conductances determined over the time course shown. Cells were exposed to isotonic medium without niflumic acid, then switched to bath medium with 100 μM niflumic acid, which produced an increase in conductance. Washout of niflumic acid (returning the cells to niflumic acid-free isotonic medium) caused the conductance to fall to control levels and finally, re-addition of niflumic acid caused the conductance to increase again. Thus, the effects of niflumic acid are reversible as would be predicted if it acts directly on an ion channel to increase conductance.

FIG. 10B shows the reversal potentials determined from the same I/V plots used to calculate conductances shown in FIG. 10A. For the given conditions of this experiment, the calculated reversal potentials for Cl and K are −22 mV and −82 mV, respectively. The actual reversal potential observed in the presence of niflumic acid is very close to the Cl reversal potential, indicating that niflumic acid activates a Cl channel to produce the increase in conductance. For the experiment shown, [Cl] and [K] in the bath medium were 102.5 mM and 5.8 mM, respectively, while [Cl] and [K] in the pipette were 42.9 mM and 150.0 mM, respectively. The electrophysiology protocol used was the same as that described above for FIG. 8.

The foregoing description of the invention is exemplary for purposes of illustration and explanation. It should be understood that various modifications can be made without departing from the spirit and scope of the invention. Furthermore, all publications, Genbank references, patents, patent applications cited herein are hereby expressly incorporated by reference for all purposes. Accordingly, the following claims are intended to be interpreted to embrace all such modifications.

1. A method for increasing aqueous humor outflow in an eye of a mammalian patient, said method comprising the step of:
administering to said eye a composition comprising an effective amount of a compound that activates an ion channel selected from the group consisting of a Cl channel, a K channel, and any combination thereof in Canal of Schlemm endothelial cells of the eye.

2. The method of claim 1, wherein the method further comprises administering to the eye an effective amount of a compound that inhibits a Na⁺—K⁺—2Cl⁻ co-transporter, said compound being the same compound or a different compound from the Cl channel or K channel activating compound.

3. The method of claim 1, wherein the compound activates both the Cl channels and the K channels.

4. The method of claim 1, wherein the compound also activates the ion channels in trabecular meshwork cells.

5. The method of claim 1, further comprising activating an ion channel selected from the group consisting of a Cl channel, a K channel, and any combination thereof in trabecular meshwork cells of the eye.

6. The method of claim 1, wherein the compound is selected from the group consisting of non-steroidal anti-inflammatory agents.

7. The method of claim 6, wherein the compound is selected from the group consisting of niflumic acid, flufenamic acid, and any combination thereof.

8. The method of claim 1, wherein the composition is administered by microinjection.

9. The method of claim 1, wherein the composition further comprises a pharmaceutically acceptable carrier.

10. The method of claim 1, wherein the composition is administered topically.

11. The method of claim 10, wherein the composition further comprises a compound that enhances corneal penetration.

12. The method of claim 11 wherein the composition further comprises 0.025% benzalkonium chloride.

13. The method of claim 10, wherein the compound is selected from the group of lipophilic derivatives of niflumic acid and flufenamic acid.

14. The method of claim 111 wherein the compound has an octanol:water coefficient of at least 0.005.

15. The method of claim 11 wherein the compound has an octanol:water coefficient of at least 0.01.

16. The method of claim 2, wherein the Na⁺—K⁺—2Cl⁻ inhibiting compound is selected from the group consisting of a benzmetamide; a bumetamide; a furosemide; a torasemide; a piretamide; a lipophilic derivative of benzmetamide, bumetamide, furosemide, torasemide, or piretamide; and any combination thereof.

17. The method of claim 1, wherein the patient is human and administration of the composition is used to lower intra-ocular pressure.

18. The method of claim 17, wherein the method of lowering intra-ocular pressure is used to treat glaucoma.

19. A method for screening compounds for utility in increasing aqueous humor outflow comprising the steps of:
a. contacting Schlemm's canal endothelial cells or trabecular meshwork cells with a compound in the presence and absence of K and/or Cl channel blockers;
b. observing physiological changes in the cells as indicative of the compound's use in regulating aqueous humor outflow.

20. The method of claim 19, wherein the observed physiological change is a change in conductance of the cells.

21. The method of claim 19, wherein the observed physiological change is a change in volume of the cells.
